(19) United States
(12) Patent Application Publication
(10) Pub. No.: US 2006/0235371 A1
(43) Pub. Date: Oct. 19, 2006

(54) Microscopic-spots irradiating device applying a vacuum thereto

(76) Inventors: Shingo Wakamatsu, Tokyo (JP); Kunihiko Mitsuda, Tokyo (JP)

Correspondence Address: EDWARDS & ANGELL, LLP, P.O. BOX 55874, BOSTON, MA, 02205, US

(21) Appl. No.: 11/376,939

(22) Filed: Mar. 16, 2006

(30) Foreign Application Priority Data
Apr. 7, 2005 (JP) JP 2005-110791
May 30, 2005 (JP) JP 2005-156874

(51) Int. Cl. A61B 18/18; A61N 1/30

(52) U.S. Cl. 606/9; 604/20

(57) ABSTRACT

The present invention relates to a microscopic-spots irradiating device applying a vacuum thereof that is provided with a laser beam or flash-lamp light-generating device, a multiple microscopic-spot generating device that creates multiple microscopic spots in cutaneous tissues, and a suction device that sucks up cutaneous tissues during the illumination thereof by the laser beam or flash lamp. The irradiating device of the present invention performs a laser treatment by forming a large number of microscopic spots in subcutaneous tissues that have been pulled up and stretched by suction, making it possible to encourage the treatment of pigmented lesions and regenerate new skin, while minimizing post-inflammatory or post-operative hyperpigmentation.

### BACKGROUND OF THE INVENTION

The present invention relates to a microscopic-spots irradiating device applying a vacuum thereto (hereafter called as “a laser treatment device”) that is used in the irradiation of lights or laser lights through the microscopic spots of micron dimensions in a skin surface by vaporization and coagulation of human skin that has been placed in contact with a laser or light treatment during treatment of the skin.

Recent increases in the working population of women and also the aging society have led to an unbounded desire for feminine beauty and rejuvenation. Over the past few years, esthetic methods that address this demand have attracted much attention as cosmetic medicine, not only in the field of cosmetics, but also in the field of medical treatment.

The devices that are most often used in the cosmetic medical treatment field are light treatment appliances that use lasers or flash lamps. There is a wide variety of these laser or flash-lamp appliances for cosmetic medical treatment in use, which differ in details such as duration time and the lasing wavelengths that are absorbed by the structure or coloration of the target tissues being treated. One theory of laser treatment for pigmentary skin diseases, in which factors such as laser wavelength and pulse duration time are adjusted, is called selective photo-thermolysis.

An essential concept of the principle of cosmetic treatment by lasers is that normal tissues that are the target tissues among the cutaneous tissues should be subjected to the optical thermal action of the laser, but the properties of these normal tissues, such as the characteristic coloring and structure or water content thereof, should not be damaged thereby. A dye laser is used if the target is hemoglobin in cutaneous tissues, an alexandrite laser is used if the target is melanin, or an erbium YAG laser is used if the target is water, as appropriate. The stimulus of the thermal action of each laser beam causes the removal of target tissues by coagulation, the respective normal cells within the cutaneous tissues repeatedly divide, and this causes the skin to repair itself. During this time, the old tissues and the replaced new tissues become rejuvenated tissues, from both the visible and histological viewpoints (see FIG. 7).

Since most lasers in current use perform treatments at high power levels and also with large spots over skin, healthy tissues other than the target tissues are subjected to thermal damage. When a laser is irradiated onto skin, the laser energy is absorbed by the melanin pigment, the melanosome is destroyed by the emitted thermal energy, and the melanin cells are also damaged thereby. In addition, if conducted heat also causes thermal damage in dermal cells and collagen fibers in the vicinity, there will scarring and post inflammatory hyperpigmentation of the skin.

For that reason, it requires a few months for the skin to recover from the acute injury and postinflammatory hyperpigmentation after each laser treatment and it is necessary to cover the site of the treatment, such as the face, with gauze dressings and/or skin-tone tape. In addition, if the melanocytes that are pigment-generating cells are damaged over a wide area, problems will occur in that the skin will be locally bleached and dermal scar formation could cause cicatricial leukoderma.

The laser treatment device that has been developed most recently in the USA is called “Fraxel”. This is a system of treatment by which a laser fiber or a scanner is used to open up several thousand microscopic coagulative spots of a diameter of 70 μm to 100 μm (microns) and at a spacing of 100 μm to 300 μm per one square centimeter of skin (refer to http://www.reliant-tech.com/science/defaul.asp).

This Fraxel device uses an erbium YAG laser that lases at a wavelength of 1550 nm. The substance that selectively absorbs this erbium laser is water. The effects obtained by irradiating this laser onto the human body apply to the water content of the irradiated tissues, not the selective reaction on human target tissues that are included with the previously described types of laser, and this action is non-selective thermal coagulation of tissues. The transmissivity of this light is directly that which occurs on passing through water, so there is little scattering to the surroundings. This action is similar to that of simple thermal coagulation caused by electric cautery that is used in ordinary surgical techniques. The depth to which this non-selective thermal coagulation action within a single laser spot is approximately 100 μm (the depth of the hole is between 100 μm to 700 μm within the skin tissues from the skin surface). An advantage of this system is that each laser spot is small, which means that the stem cells and melanocytes of the dermal papillary layer of the subcutaneous tissues surrounding the area affected by each laser beam are largely undamaged. This causes rapid rejuvenation of the epidermis from the surrounding spared normal skin tissues, enabling rejuvenation of the outer surface of the skin. For that reason, it is best that the irradiated microscopic spots in the skin surface are as small as possible.

However, since this Fraxel device uses an erbium laser that has non-selective absorption characteristics with respect to cutaneous tissues, there are mechanical limits on the formation of the laser spot size, and the company recommends six to eight treatments within one or two weeks to achieve a certain treatment effect, and also the thermal coagulation action extends over a wide area of the skin in general, it can cause micro-scarring and hyperpigmentation in people with oriental skin such as Japanese.

A conceptual view of this type of prior-art laser treatment device is shown in FIG. 8. In sequence within a sleeve 30, a laser beam from a laser source strikes a lens 10 and is focused thereby, then passes through multiple microscopic-spot generating device 20 to form laser beams of a spot size on the order of 30 to 50 μm in diameter. These beams irradiate human skin to pierce therethrough and thereby form microscopic spots ho by vaporization by the laser beams in the epidermal layer and dermal layer (see FIG. 7). However, with this type of laser treatment device, the spot size of the laser beams is limited so it is extremely difficult to form microscopic spots in the skin of a diameter of approximately 30 μm or less.

Furthermore, since this Fraxel device uses a micro-processing configuration, the price thereof is extremely high at between several million yen (87,000 US dollars) to 16 million yen (139,000 US dollars), which is a factor in increasing patient treatment charges.

Another anti-aging medical treatment device that has been developed in the USA recently is called Aesthera PPx technology, which involves pulling up skin and irradiating it with light. With this method, light from a powerful flash lamp, not a laser, is shone onto the skin while the skin is sucked upward, with the melanin and follicles that are the targets in the subcutaneous tissues being irradiated as close to the skin surface as possible (refer to http://www.aesthera.com/default/index.cfm).

Since this Aesthera PPx technology like other flash lamp technologies selects and damages targets that absorb a certain wavelength of light, the targets of this treatment are mainly wrinkles, melanin in hair or the like, and hemoglobin that forms capillary dilatations, although it is designed with the objective of rejuvenation such as the regeneration of cutaneous tissues, there are much needs for the higher power of treatment device with minimal damage to the skin.

The present invention addresses the above problems by using kinds of laser beams that have selective pigment absorbability and destructibility which is not possible with the Fraxel and Aesthera PPx conventional technology. Pigmented lesions can be treated efficiently and also the effects of this device can be restricted to a superficial area since the penetration depth of the effective energy into the skin by the irradiated any lights thorough a small hole is limited only to the very superficial layer of the skin which spares any damage to the underlying collagen tissue so that post-inflammatory hyperpigmentation is prevented. This new invention of laser device that has a screening plate which has been readily drilled with microscopic holes and which is affixed to the skin surface and is irradiated with the laser. The characteristics of this laser device are both selective pigment absorbability (such as melanin tissues of wrinkles) and restricted depth of penetration of light locally through the microscopic holes.

### SUMMARY OF THE INVENTION

A microscopic-spots irradiating device applying a vacuum thereto in accordance with the present invention comprises a laser beam or flash-lamp light-generating device, a multiple microscopic-spot generating device that creates a large number of microscopic spots in cutaneous tissue, and a suction device that sucks up the cutaneous tissue during the irradiation thereof by the laser beam or flash-lamp light. By using light from the laser beam or flash lamp through microscopic spots by irradiation in subcutaneous tissues that have been sucked upward, this device enables the formation of microscopic spots that have a smaller diameter when the subcutaneous tissues are returned to their original position, thus enabling prevention of minimizing the epidermal barrier function for rejuvenation of new tissues.

### BRIEF DESCRIPTION OF THE DRAWINGS

FIG. 1A is a conceptual view of an embodiment of the microscopic-spots irradiating device applying a vacuum thereto (hereafter called as “the laser treatment device”) in accordance with the present invention, and FIG. 1B is a conceptual view of another embodiment of the laser treatment device of the present invention which is further provided with a medication spray device;

FIG. 2A is a vertical section through the laser treatment device of the present invention in a state in which the suction device has been moved and the skin has been stretched, and FIG. 2B shows a state in which the connection to the vacuum source has been disconnected and the suction space has returned to atmospheric pressure.

FIG. 3 is a vertical section through the laser treatment device of the present invention which is provided with a liquid medication spray device;

FIGS. 4A to 4C show the microscopic-spot generating device used in the laser treatment device of the present invention, where FIG. 4A is a perspective view of a film for laser treatment which is formed by creating a large number of microscopic holes in a thin film and which is used as the multiple microscopic-spot generating device, FIG. 4B is a perspective view of a film for laser treatment that is circular in a plan view, and FIG. 4C is a section taken along the line I-I of FIG. 4A.

FIG. 5A is a perspective view of another embodiment of the microscopic-spot generating device used in the laser treatment device of the present invention, with FIG. 5B being a plan view thereof;

FIG. 6 shows a state in which a thin film that is used as the microscopic-spot generating device of the laser treatment device of the present invention is placed on the surface of human skin and is irradiated with a laser that passes through microscopic holes in that film, to open up spots in the epidermal layer and dermal layer;

FIG. 7 shows a section through the structure of human skin and the method of using the laser treatment device to form microscopic spots in cutaneous tissues; and

FIG. 8 is a vertical section through a laser treatment device of the prior art.

### DETAILED DESCRIPTION OF PREFERRED EMBODIMENTS

### Embodiment 1

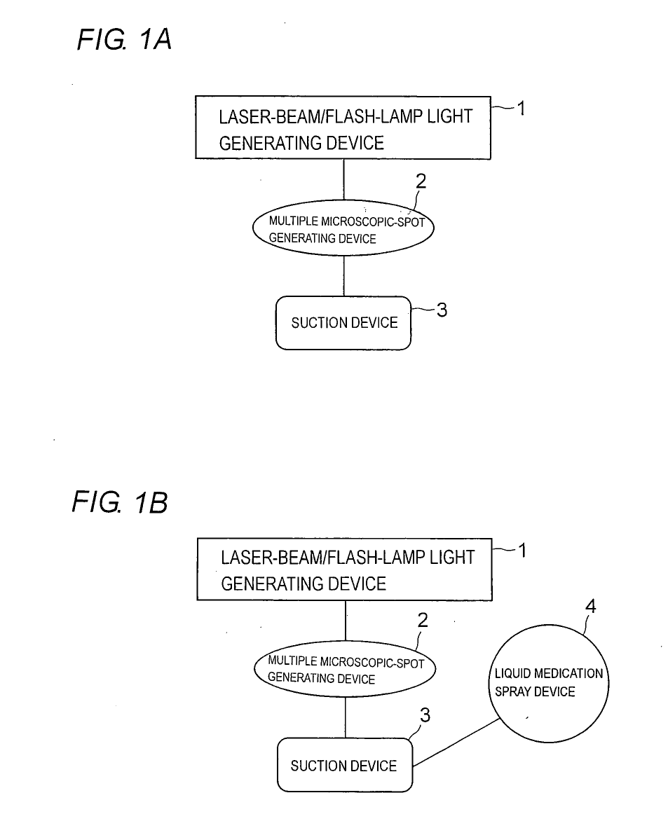

A conceptual view of an embodiment of the laser treatment device of the present invention is shown in FIG. 1A. The laser treatment device of the present invention consists of a laser-beam/flash-lamp light-generating device 1, a multiple microscopic-spot generating device 2 that causes the light of the laser beam or flash lamp from the laser-beam/flash-lamp light-generating device 1 to pass through a large number of microscopic spots that have been pierced through a plate-shaped body, and a suction device 3 that uses vacuum suction to stretch the epidermal layer of skin during the illumination thereof by the laser beam or flash lamp.

In addition to the basic configuration of FIG. 1A, a liquid medication spray device 4 that sucks up a liquid medication and injects it could also be connected to the suction device 3, to suck up and spray a predetermined liquid medication onto the skin when the pressure within the suction device 3 is returned to atmospheric pressure from the negative pressure, as shown in FIG. 1B.

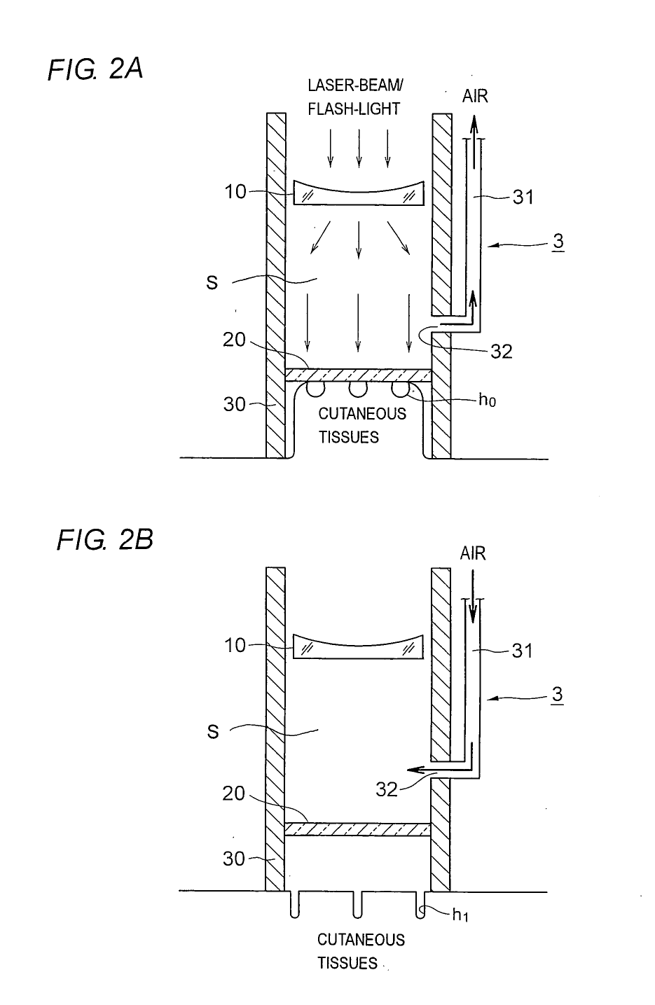

A specific configuration of the laser treatment device in accordance with the present invention will now be described, with reference to FIGS. 2A and 2B.

As shown in FIG. 2A, the laser treatment device of the present invention is provided with a sleeve 30 within which are arranged a lens 10 that deflects and focuses the light of the laser beam or flash lamp from the light source; a multiple microscopic-spot generating device 20 therebelow which is in contact with the skin when it is sucked up, to form microscopic spots therein; and the suction device 3 which is connected to a vacuum source and which also penetrates through the sleeve 30 and is connected thereto dynamically.

As further shown in FIG. 2A, the suction device 3 is connected to a suction space S by a suction tube 31 that passed through the sleeve 30; an aperture portion 32 formed at one end of the suction tube 31 opens into the suction space formed between the lens 10 and the multiple microscopic-spot generating device 20 housed in the sleeve 30; and the other end of the suction tube 31 is connected to a predetermined vacuum source (such as a vacuum pump) to evacuate the interior of that suction space.

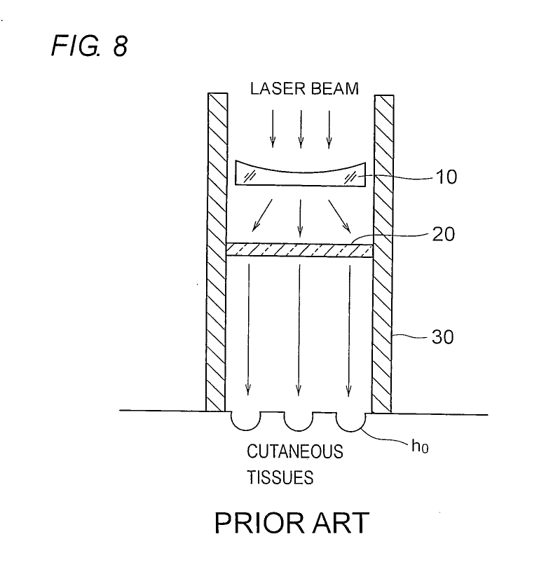

As previously stated, there is a physical limit on the size (diameter) of the spots formed in the skin (living tissues) by the action of the laser beam (such as vaporization) with the conventional laser treatment device shown in FIG. 8, since the laser beam passes only through the multiple microscopic-spot generating device 20. In this case, the “vaporization” of living tissues refers to a process of using a laser that has a high rate of absorption by water (such as a CO₂ laser). Human tissues are approximately 65% water and most of the laser energy that is incident on the living tissues is converted into thermal energy and is absorbed by the epidermal layer of the skin. This causes an explosion of steam that is said to destroy the living tissues.

With the laser treatment device of the present invention shown in FIG. 2A, the suction space S formed during the laser illumination between the lens 10 and the multiple microscopic-spot generating device 20, from the suction tube 31 connected to the vacuum source, forms a suction pressure of 13.790 to 41.370 kiloPascals (2 to 6 psi). When the skin is pulled up by this suction until it comes into contact with the surface of the multiple microscopic-spot generating device 20 and is irradiated by the laser beam in this state, vaporization spots ho of the same diameter as that of the microscopic spots of the microscopic-sot generating device are formed in the stretched subcutaneous tissues, as shown in FIG. 2A.

After the laser illumination, the connection to the vacuum source is broken, returning the atmosphere from the suction tube 31 and through the aperture portion 32 so that the suction space S returns to atmospheric pressure, as shown in FIG. 2B. This returns the stretched skin to its original form, the spots ho that have been formed in the subcutaneous tissues by vaporization are compressed to form microscopic spots h₁ of a smaller diameter, so that a large number of microscopic spots of a diameter that is suitable for laser treatment of the skin (such as 30 μm or smaller) are formed in the skin.

Experiments and documentation produced by the present inventors (refer to “The optics of stretching skin and use during clinical laser treatments”, Steven L. Jacques, Vic A Narukar MD, Robert Anderson, Progress Report, prepared for distribution at the American Academy of Dermatology) has shown that when skin is sucked up by the suction device 3, the skin is pulled upwards and stretched by about 25% to 35%. Thus, if microscopic spots of a diameter of 50 μm that have been formed in the skin by vaporization during the suction phase are returned to atmospheric pressure, this would be the same as forming microscopic spots of a diameter on the order of 37 μm to 40 μm in the skin. Thus, since the laser treatment device of the present invention enables the formation of a large number of microscopic spots with little damage to subcutaneous tissues, it promotes the rejuvenation of new skin without completely destroying the melanocyte cells of the subcutaneous tissues. As a result, it is possible to achieve a rejuvenation effect throughout the human skin.

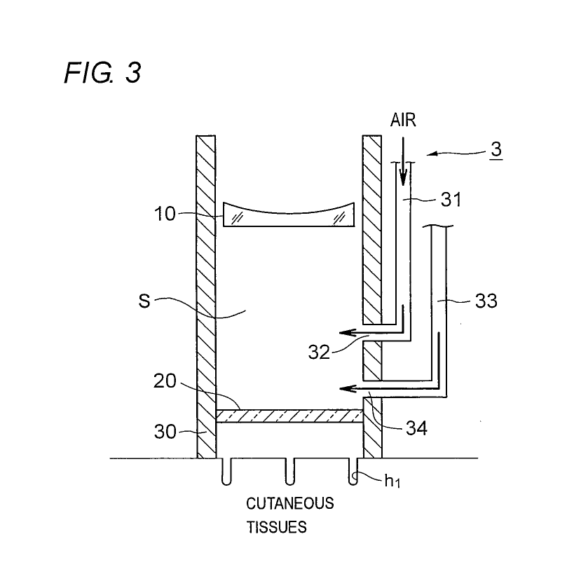

In addition, a liquid medication spray tube 33 that is connected to a liquid medication source is connected to the sleeve 30, as shown in FIG. 3, and the liquid medication spray device 4 (See FIG. 1B) is operated during the return from the negative pressure to atmospheric pressure to deliver a liquid medication through a liquid medication spray port 34 into the suction space S. Furthermore, since the suction space S is in a negative pressure state during the suction, the liquid medication is automatically pulled into the suction space S on the return to atmospheric pressure. This enables ample application of the medication and the permeation thereof into the skin, in a simple manner.

Note that this type of medication includes vitamin C and derivatives thereof that aid in the activation of cutaneous tissues, vitamin E or the like, and hyaluronic acid that is used in applications such as the latest anti-aging treatment, by way of example.

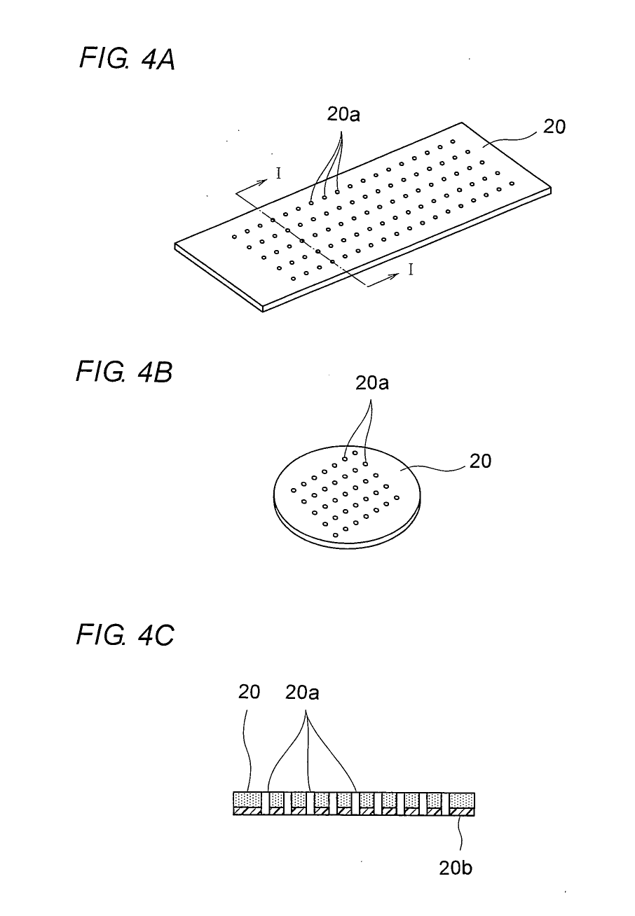

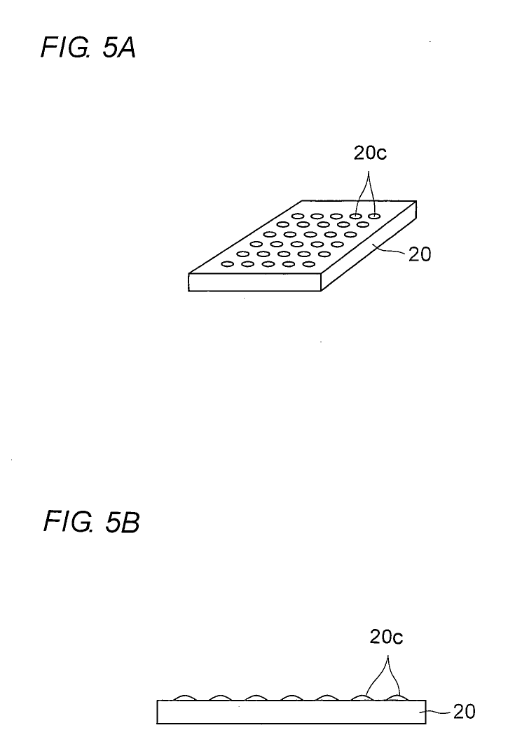

Perspective views of a film used in the microscopic-spot generating device of the laser treatment device of the present invention (hereinafter called “film”) are shown in FIG. 4A to FIG. 4C, where FIG. 4A shows a film in a thin plate shape (rectangular) and FIG. 4B shows a circular film. This film 20 is formed of an elastic sheet material of suitable dimensions through which the laser cannot pass, in a thin plate shape (FIG. 4A) or circular shape (FIG. 4B). A large number of microscopic spots 20a of a diameter of 1 nm (nanometer) to 1000 μm (microns) are provided through this sheet.

In this case, the sheet material is formed to any desired dimensions, such as circular, square, or oval; the size of the microscopic holes 20a can be set such that the diameter thereof is within the range of 1 nm (nanometer) to 1000 μm (micrometers or microns); and the size and number of the microscopic holes 20a per unit area can be varied in accordance with the objective of the skin treatment and the operator's selection. As a standard, the size of the microscopic holes is set to 30 μm to 500 μm, depending on pathological necessity, and the microscopic holes 20a are pierced through the film at a spacing of 50 μm to 500 μm, depending on the shape and size of the film. In addition, adhesive 20b (such as a silicone-based adhesive or hypoallergenic acrylic-based adhesive) could be coated on one surface of the film 20 as shown in FIG. 4C (in which the size of the microscopic holes 20a is exaggerated) so that the film 20 can be affixed to the skin.

Furthermore, medication (such as vitamin C, retinoic acid, or an antioxidant) or adhesive could be coated onto the surface of the film 20 that comes into contact with the skin, or medication could be permeated therein.

Still further, a marker that changes color when subjected to the heat of the laser (such as carbon powder) could be coated onto a portion of the surface of the film 20 that is irradiated by the laser, in order to provide visual confirmation that that portion has been irradiated by the laser.

Even further, the multiple microscopic-spot generating device could be configured by drilling a large number of the microscopic holes through a plate-shaped body 20 formed of a predetermined material (such as glass or plastic), then dropping a fixed quantity of melted glass or plastic onto the aperture of each of those microscopic holes so that the surface tension thereof is utilized to form a lens body 20c.

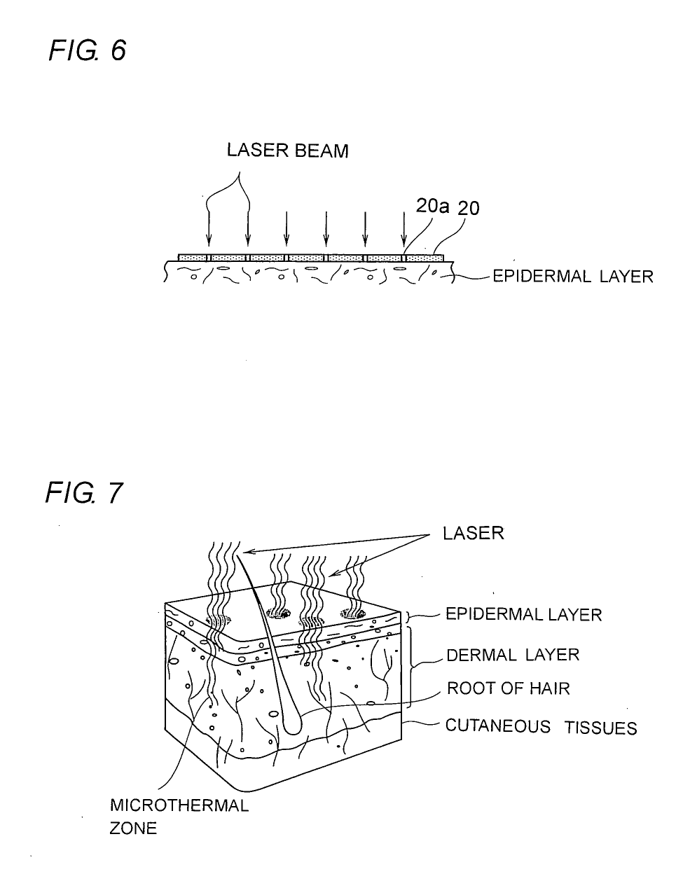

When a laser treatment is performed on human skin, using the thus-constructed film 20 for laser treatment in accordance with the present invention, the film 20 of one of these thin plate shapes is placed on or affixed to the skin surface (epidermal layer), suction is applied by the suction device 3 of FIG. 2A, and the laser is shone onto this film 20 from above, as shown in FIG. 6. When this happens, the laser passes through the microscopic holes 20a in the film 20, making it possible to open up a large number of microscopic holes that are smaller (such as 30 μm to 300 μm) than the spots created by simply placing a conventional laser device on the skin surface.

When light having a luminous flux of a usual diameter has been shone onto a dispersed material such as skin, the diameter d of the luminous flux that retains an effective energy at a certain depth of the skin is generally given by the following equation:
(diameter d of luminous flux at depth h (cm) of skin)=(diameter d₀ of luminous flux at skin surface)·(depth h (cm) of skin)/2

In other words, it is clear from the above equation that the depth to which the effective energy penetrates within the subcutaneous tissues can be freely controlled by changing the size of the spots drilled in the skin as required.

Since the film for laser treatment in accordance with the present invention is based on this principle, it is possible to perform laser treatment while minimizing damage to tissues that do not require treatment, by combining this film with the characteristics of an existing laser device.

As described above, it is also possible to make medication permeate as far as the epidermal layer in a simple manner, by painting medication or the like on the skin immediately after the laser treatment or by using the film of the present invention that has been soaked in the medication.

Furthermore, it is possible to make use of existing surgical devices and laser processing devices that open microscopic spots in skin and other parts of human bodies, but to a smaller diameter than that of the microscopic spots that can be created by these existing laser treatment devices, by pulling up and stretching the skin during the irradiation by light from the laser or flash lamp, to create a large number of microscopic spots.

What is claimed is:

1. A microscopic-spots irradiating device applying a vacuum thereto comprising:
a laser beam or flash-lamp light-generating device;
a multiple microscopic-spot generating device for forming a large number of microscopic spots in cutaneous tissues; and
a suction device for sucking up cutaneous tissues during the illumination by light thereof from said laser beam or flash-lamp.

2. The irradiating device according to claim 1, further comprising: a medication spray device for spraying a liquid medication on cutaneous tissues after the illumination thereof

3. The irradiating device according to claim 1, wherein: said multiple microscopic-spot generating device is formed from a plate-shaped body or a round plate pieced with a large number of microscopic holes.

4. The irradiating device according to claim 1, wherein: said multiple microscopic-spot generating device is formed of a plate-shaped body of aluminum, glass, or plastic through which a large number of microscopic holes are formed, where glass or plastic is melted into an aperture portion of each of said microscopic hole, to form a lens body.

5. The irradiating device according to claim 1, wherein: the medication that is sprayed by said medication spray device comprises vitamin C or a derivative thereof, vitamin E, or hyaluronic acid.

6. The irradiating device according to claim 1, wherein: said multiple microscopic-spot generating device has a large number of microscopic holes that are formed to penetrate a thin film at a predetermined spacing.

7. The irradiating device according to claim 6, wherein: said film for laser treatment is rectangular in a plan view.

8. The irradiating device according to claim 6, wherein: said film for laser treatment is circular in a plan view.

9. The irradiating device according to claim 6, wherein: the shape of said microscopic holes formed in said film is circular, square, or oval.

10. The irradiating device according to claim 6, wherein: the diameter of said microscopic holes formed in said film is between 1 nm and 1000 μm, and is preferably no more than 30 μm.

11. The irradiating device according to claim 6, wherein: a medication and/or adhesive is coated onto or permeated into a surface of said thin film.

12. The irradiating device according to claim 6, wherein: a marker that changes color when a laser beam is applied thereto is coated on a surface of said thin film.

13. The irradiating device according to claim 6, wherein: said thin film is formed of a metal such as aluminum, a plastic material, paper, rubber, or cloth.
